(19) United States
(12) Patent Application Publication
(10) Pub. No.: US 2017/0360324 A1
(43) Pub. Date: Dec. 21, 2017

(54) ELECTRIC WAVE TYPE BIOSENSOR

(71) Applicant: Shigetomo Mitani, Aichi (JP)

(72) Inventor: Shigetomo Mitani, Aichi (JP)

(73) Assignee: OMRON AUTOMOTIVE ELECTRONICS CO., LTD., Aichi (JP)

(21) Appl. No.: 15/629,410

(22) Filed: Jun. 21, 2017

(30) Foreign Application Priority Data
Jun. 21, 2016 (JP) 2016-122691

(51) Int. Cl. A61B 5/05; A61B 5/00

(52) U.S. Cl. CPC A61B 5/05; A61B 5/7278; A61B 5/6887; A61B 5/7221

(57) ABSTRACT

An electric wave type biosensor includes: an electromagnetic wave irradiation unit; and a reflected wave receiving unit which receives a reflected wave and obtains an I signal and a Q signal. The electric wave type biosensor further includes: an I-Q norm angular velocity calculation unit which calculates an angular velocity and an IQ norm of the I signal and the Q signal, based on the I signal and the Q signal; a bio-information extract unit which extracts bio-information of the living body, based on the calculated angular velocity; and an output determination unit which determines whether the bio-information extracted by the bio-information extract unit is to be output based on whether a size of the calculated angular velocity is within a first threshold value.

### CROSS-REFERENCES TO RELATED APPLICATIONS

This application is based upon and claims the benefit of priority from Japanese Patent Application No. 2016-122691, filed on Jun. 21, 2016, the entire contents of which are incorporated herein by reference.

### FIELD

One or more embodiments of the present invention relate to an electric wave type biosensor that uses a Doppler sensor.

### BACKGROUND

From the related art, a technology which irradiates a human body surface with an electromagnetic wave by using a Doppler sensor and obtains bio-information included in a reflected wave based on a coordinate plane configured of an I signal and a Q signal of the reflected wave, is known. For example, JP-A-2006-055504 discloses a heart rate measuring apparatus which detects an output signal including an amplitude component and a phase component of a reflected wave from a human body surface by using an electric wave type Doppler sensor, and extracts only a heart rate component by separating the amplitude component generated by body movement of a human body. The heart rate measuring apparatus outputs an amplitude component signal and a phase component signal to a heart rate extractor by performing polar coordinate conversion using an amplitude and phase converter with respect to an output signal (an I signal and a Q signal) including information of the amplitude component and the phase component of the reflected wave output by the electric wave Doppler sensor. The heart rate extractor extracts only accurate heart rate by separating the amplitude component generated by the body movement included in the amplitude component output from the amplitude component signal and the phase component signal by using a method of independent component analysis.

In addition, JP-A-2010-120493 discloses a bio-signal sensing apparatus which prevents deterioration of accuracy of a bio-signal of an occupant. The bio-signal sensing apparatus includes: a sensor unit which senses movement of the occupant by an electric wave type non-modulation Doppler sensor; a bio-signal extract unit which extracts a bio-signal of the occupant based on a phase change of an output of the sensor unit; a distance calculation unit which calculates an estimated distance between the sensor unit and the occupant based on an integrated value of a phase change amount of the output of the sensor unit; and a bio-signal output determination unit which determines reliability of the bio-signal based on the estimated distance and stops the output of the bio-signal in a case where the reliability is low.

The sensor unit includes a local oscillator, a transmission antenna, a receiving antenna, a distributor, or a mixer, and a transmission signal is radiated toward a driver. A local signal T(t) having a frequency fHz expressed by, for example, T(t)=cos(2πft) is emitted from the local oscillator, and a part of the emitted electric wave is reflected and received by the receiving antenna as a receiving signal R(t) approximated by R(t)=cos(2πft=4πd(t)/λ−4πx(t)/λ) (wherein d(x) is a distance displacement between the sensor unit and the driver, x(t) is a fine distance displacement of a body surface including heart rate or respiration of the driver, and λ is a wavelength of the local signal T(t)).

The receiving signal R(t) is distributed into two by the distributor and input into two mixers. In addition, one more local signal T(t) distributed by the distributor is distributed into two in a state where only one phase is shifted by π/4 radian by the distributor, and is input into each of two mixers, and the local signal T(t) and the receiving signal R(t) are mixed with each other. A base band component which is close to a DC region and a modulation component are output by a multiplication operation in the two mixers, but as each of the output signals passes through a low pass filter, a real part Bi(t) and an imaginary part Bq(t) which are expressed as follows in the base band receiving signal including only the base band component, are obtained.

Bi(t)=1/2 cos(4πd(t)/λ+4πx(t)/λ)

Bq(t)=1/2 cos(π/4+4πd(t)/λ+4πx(t)/λ)

These parts are converted into a digital signal from an analog signal by an AD converter, and are input to a bio-signal extract unit as a detected signal output by the sensor unit.

In addition, JP-A-2011-015887 discloses a biological state obtaining apparatus or the like which can obtain a bio-signal of a living body in a non-contact manner, and can obtain information related to a biological state without performing complicated processing, such as frequency analysis with respect to a bio-signal. The biological state obtaining apparatus includes: an IQ signal obtaining part which transmits an electromagnetic wave to a body surface of the living body, IQ-wave-detects a reflected wave thereof, and consecutively obtains an I signal and a Q signal which are output from an IQ-wave detector that outputs the I signal and the Q signal in a time series; and a biological state obtaining part which obtains a state of the living body based on a trajectory on an IQ plane of an obtained signal obtained by the IQ signal obtaining part.

In addition, JP-A-2014-039838 discloses a biological state obtaining apparatus or the like which can obtain a bio-signal of a living body in a non-contact manner, and can obtain information related to a biological state without performing complicated processing, such as frequency analysis, with respect to a bio-signal. The biological state obtaining apparatus transmits an electromagnetic wave to a body surface of the living body, IQ-wave-detects a reflected wave thereof, consecutively obtains an I signal and a Q signal in a time series, and obtains a state of the living body based on a trajectory on an IQ plane of an obtained signal. The biological state obtaining apparatus extracts a heart rate signal from time series data of a norm of a position vector of the obtained signal on an IQ plane, detects the heart rate signal which corresponds to one heart rate based on periodic fluctuation of a waveform of the extracted heart rate signal, and calculates the heart rate in a unit period as heart rate information.

### SUMMARY

However, in the above-described related art, in a case where a large fluctuation is generated on the body surface of the living body as the human body moves, there is a case where wrong bio-information is output.

One or more embodiments of the invention provide an electric wave type biosensor which can output normal bio-information in the electric wave type biosensor that uses a Doppler sensor.

According to one or more embodiments of the invention, there is provided an electric wave type biosensor including: an electromagnetic wave irradiation unit which irradiates a body surface of a living body with an electromagnetic wave; a reflected wave receiving unit which receives a reflected wave obtained as the electromagnetic wave irradiated by the electromagnetic wave irradiation unit and then reflected on the body surface, and obtains an I signal obtained by multiplying the irradiated electromagnetic wave signal and the received reflected signal, and a Q signal obtained by delaying the I signal by a predetermined phase; an I-Q norm angular velocity calculation unit which calculates an angular velocity and an IQ norm of the I signal and the Q signal, based on the I signal and the Q signal which are obtained by the reflected wave receiving unit; a bio-information extract unit which extracts bio-information of the living body, based on the angular velocity calculated by the I-Q norm angular velocity calculation unit; and an output determination unit which determines whether or not the bio-information extracted by the bio-information extract unit is output, based on whether or not a size of the angular velocity calculated by the I-Q norm angular velocity calculation unit is within a first threshold value.

According to this, it is possible to provide an electric wave type biosensor that can output accurate bio-information by stopping the output of the bio-information in a case where a large fluctuation is generated on the surface of the living body.

In the electric wave type biosensor, after determining that the bio-information is not output, the output determination unit may determine that the bio-information extracted by the bio-information extract unit is to be output in a case where the IQ norm calculated by the I-Q norm angular velocity calculation unit is within a second threshold value, and the size of the angular velocity calculated by the I-Q norm angular velocity calculation unit is within the first threshold value.

According to this, by restarting the output in a case where the IQ norm is within a predetermined threshold value, it is possible to output the accurate bio-information.

In the electric wave type biosensor, an estimating unit which estimates the size of the angular velocity based on data of the angular velocity of a time series which is calculated by the I-Q norm angular velocity calculation unit, may further be provided, and the output determination unit may determine whether or not the bio-information extracted by the bio-information extract unit is output, based on the size of the angular velocity estimated by the estimating unit and based on whether or not a size of the estimated angular velocity is within the first threshold value.

According to this, by estimating whether or not the displacement of the surface of the living body deviates from the range that can be measured, it is possible to rapidly determine whether or not the output is performed, and to output only the accurate bio-information without outputting wrong bio-information.

According to one or more embodiments of the invention, it is possible to provide an electric wave type biosensor that can output accurate bio-information, in the electric wave type biosensor that uses a Doppler sensor.

### BRIEF DESCRIPTION OF THE DRAWINGS

FIG. 1 is a schematic view in which an electric wave type biosensor of an embodiment of the invention is installed in a vehicle interior of a vehicle;

FIG. 2 is a block diagram of the electric wave type biosensor of the embodiment of the invention;

FIG. 3 is a block diagram of a Doppler sensor in the electric wave type biosensor of the embodiment of the invention;

FIG. 4 is a view describing an estimating method of an estimating unit of the electric wave type biosensor of the embodiment of the invention;

FIG. 5 is a view describing restart of an output in an output determination unit of the electric wave type biosensor of the embodiment of the invention;

FIG. 6 is a flowchart illustrating a control in the electric wave type biosensor of the embodiment of the invention; and

FIG. 7 is a view for describing an angular velocity or the like on an I-Q coordinate plane.

### DETAILED DESCRIPTION

In embodiments of the invention, numerous specific details are set forth in order to provide a thorough understanding of the invention. However, it will be apparent to one of ordinary skill in the art that the invention may be practiced without these specific details. In other instances, well-known features have not been described in detail to avoid obscuring the invention.

Hereinafter, an embodiment of the invention will be described with reference to the drawings. An electric wave type biosensor according to the embodiment of the invention irradiates a human body surface with an electromagnetic wave by using a Doppler sensor, determines a case where a large fluctuation is generated on a body surface of a living body as the human body moves according to whether or not the size of an angular velocity of an I signal/Q signal exceeds a predetermined threshold value in a case of obtaining bio-information that is accompanied with fine movement included in the reflected wave, and can output accurate bio-information by stopping an output of the bio-information in the case.

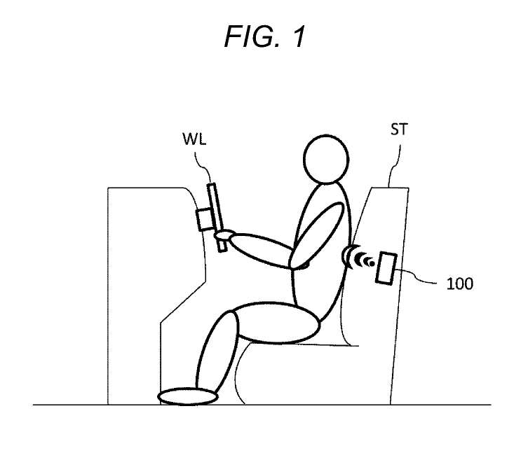

An electric wave type biosensor 100 in the embodiment will be described with reference to FIGS. 1 to 3. The electric wave type biosensor 100 is installed in equipment having a surface which is directly or indirectly in contact with a part of the human body, and senses the bio-information of a user of the equipment. Here, the equipment (general term of tool, machinery, and machine) having a surface which is in contact with a part of the human body is specifically referred to as, for example, a chair or a sofa on which a human sits, a bed on which a human lies down, a body inspection equipment installed in a hospital, and a seat which is installed in a vehicle or an airplane and on which a human sits.

A surface which is in contact with a part of the human body is referred to as a seat surface or a backrest surface in a chair or the like, and a mattress upper surface in a bed. The surface may be directly or indirectly in contact with a part of the human body, or may indirectly come into contact with the human body as a human wears clothes. A part of the body is a buttock or a thigh on a seat surface of a chair or the like, and is generally referred to as the back in the backrest of a chair or the like or in a bed or the like. In the body inspection equipment, a part of the body may be any of arms and legs of a human.

In the specification, the bio-information of the user is referred to as the size of the heart rate (pulse rate) or a pulse wave, or respiratory frequency or the size of respiration, and does not include cough or sneeze which generates movement of skin or muscle which does not come from the heart rate or respiration. The heart rate or the respiration generates fine movement on the body surface of the living body, and the electric wave type biosensor 100 detects the bio-information that is accompanied with the fine movement.

In the embodiment, a case where the electric wave type biosensor 100 is installed in an interior of a vehicle as illustrated in FIG. 1 will be described. The electric wave type biosensor 100 is installed in a backrest portion of a seat ST on which the driver or the like sits. Since the purpose of the electric wave type biosensor 100 is to sense the fine movement of the skin surface which is accompanied with the heart rate or respiration, a case where the electric wave type biosensor 100 is installed in the backrest portion which is a surface that is in contact with the back of the driver who does not have a relatively large movement is more preferable than a case where the electric wave type biosensor 100 senses the movement by irradiating a face or the like of the driver who has a large movement in a forward handle WL direction with an electric wave.

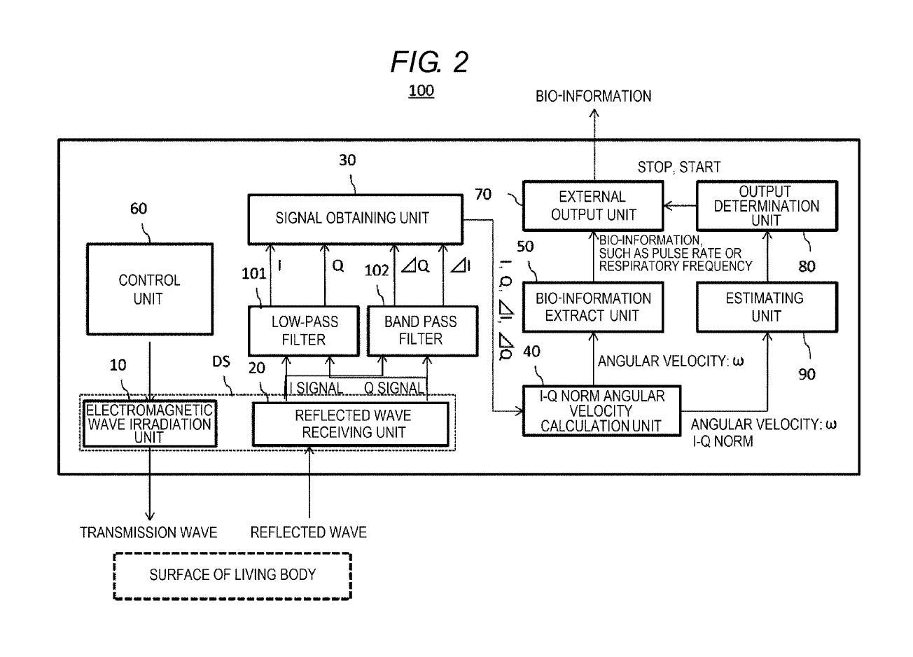

As illustrated in FIG. 2, the electric wave type biosensor 100 includes an electromagnetic wave irradiation unit 10 which irradiates the body surface of the living body with the electromagnetic wave; a reflected wave receiving unit 20 which obtains an I signal obtained by multiplying the signal of the irradiated electromagnetic wave and the received reflected signal, and a Q signal obtained by delaying the I signal by a predetermined phase, after the reflected wave obtained as the electromagnetic wave irradiated by the electromagnetic wave irradiation unit 10 is reflected on the body surface is received and wave detection or amplification is performed; and a control unit 60 which controls the electromagnetic wave irradiation unit 10. In addition, the electromagnetic wave irradiation unit 10 and the reflected wave receiving unit 20 configure a Doppler sensor DS.

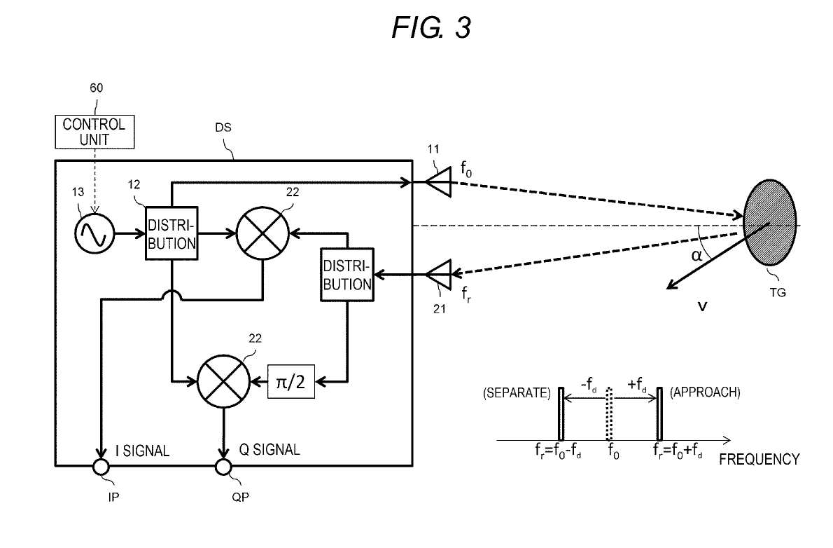

FIG. 3 is a block diagram specifically illustrating the Doppler sensor DS. An oscillator 13 of the Doppler sensor DS oscillates at a predetermined frequency by a control of the control unit 60. In addition, a microwave band of the frequency is generally used, and there are many cases where the frequency is not particularly limited, but 24 GHz is generally used in a case of a use for obtaining the bio-information. The electromagnetic wave oscillated by the oscillator 13 is distributed by a distributor 12, and a measurement target TG is irradiated with one of the electromagnetic waves as an electromagnetic wave having a frequency f₀ (for example, 24 GHz) from a transmission antenna 11.

The electromagnetic wave of the frequency f₀ is reflected abutting against the measurement target TG having a movement, the frequency changes to frequency fᵣ, and a receiving antenna 21 receives the reflected wave that becomes the frequency fᵣ. In addition, the measurement target TG moves at a relative velocity v in a direction having an intersecting angle α with respect to directions of the transmission antenna 11 and the receiving antenna 21. Then, reflected wave frequency fᵣ is acquired by the equation (1).

fᵣ=f₀±f_{d} (1)

A transmission wave frequency is f₀, a Doppler frequency is f_{d}=(2f₀|v|/c₀)·cos α, a light velocity is c₀, a relative movement velocity of the measurement target is v, and an intersecting angle in the moving direction of the measurement target with respect to the transmission wave is α.

The reflected wave of the frequency fᵣ received by the receiving antenna 21 is computed to be multiplied with the other electromagnetic wave (frequency f₀) distributed by the distributor 12 in a mixer 22, and is output from an I signal output port IP which is a part of the reflected wave receiving unit 20 as the I signal including a base band component that is close to a DC region and a modulation component. In addition, the reflected wave which is a reflected wave of the frequency fᵣ received by the receiving antenna 21 and of which a phase is shifted by π/2, is similarly computed to be multiplied with the other electromagnetic wave (frequency f₀) distributed by the distributor 12 in the mixer 22, and is output from a Q signal output port QP which is a part of the reflected wave receiving unit 20 as the Q signal including the base band component that is close to the DC region and the modulation component.

The electric wave type biosensor 100 further includes a low pass filter 101 and a band pass filter 102 into which the I signal output from the I signal output port IP and the Q signal output from the Q signal output port QP by the reflected wave receiving unit 20 are input; and a signal obtaining unit 30 which obtains a signal which will be described later from each of the low pass filter 101 and the band pass filter 102. The low pass filter 101 is an arbitrary filter which removes noise of a high-frequency component and allows only the base band component to pass through in the I signal and the Q signal output by the I signal output port IP and the Q signal output port QP, and outputs signals (I and Q) that are the smoothed I signal and Q signal. In addition, since the purpose of the electric wave type biosensor 100 is to obtain the bio-information, such as heart rate or respiration, the low pass filter 101 is a filter which allows a heart rate of approximately 1 Hz or respiration of approximately 0.3 Hz to pass, and for example, is a filter which removes the heart rate or respiration which is equal to or greater than 10 Hz.

The band pass filter 102 is a selective filter which removes the DC component from the I signal and the Q signal which are output by the I signal output port IP and the Q signal output port QP, and outputs differential values (ΔI and ΔQ) of each signal.

The signal obtaining unit 30 receives the I signal and the Q signal of which the high-frequency component is removed by the low pass filter 101, and an I signal differential value ΔI which is a differential value of the I signal from the band pass filter 102 and a Q signal differential value ΔQ which is a differential value of the Q signal. In addition, the signal obtaining unit 30 may be an AD port including an AD converter that converts an analog signal into a digital signal and is mounted in a microcomputer. In addition, configuration elements, such as the control unit 60 or an I-Q norm angular velocity calculation unit 40 which will be described later, may be mounted in the microcomputer.

The electric wave type biosensor 100 further includes the I-Q norm angular velocity calculation unit 40 which calculates the angular velocity of the I signal and the Q signal based on the I signal and the Q signal which are obtained by the reflected wave receiving unit 20 and the I signal differential value ΔI and the Q signal differential value ΔQ which are calculated by the band pass filter 102 based on the I signal and the Q signal. As will be described later, the I-Q norm angular velocity calculation unit 40 acquires an angular velocity ω and an IQ norm NRM of the I signal and the Q signal based on the I signal, the Q signal, the I signal differential value ΔI, and the Q signal differential value ΔQ.

A transmission wave 240 of the frequency f₀ in accordance with time t, which is transmitted by the transmission antenna 11 of the Doppler sensor DS, is expressed by the equation (2).

xₛ(t)=Aₛ cos(ωₛt) (2)

A transmission wave amplitude is Aₛ, and a transmission wave angular velocity is ωₛ=2πf₀.

In addition, a reflected wave xᵣ(t) of the frequency fᵣ in accordance with time t, which is received by the receiving antenna 21 of the Doppler sensor DS, is expressed by the equation (3).

xᵣ(t)=Aᵣ cos([ωₛ±ω_{d}]t+φ) (3)

A receiving wave amplitude is Aᵣ, a Doppler angular velocity is ω_{d}=2πf_{d}, and a phase which depends on a distance to the measurement target is φ.

In addition, a signal which is computed to be multiplied by inputting the transmission wave and the reflected wave into the mixer 22, is expressed by the equation (4).

$$\begin{matrix}{x}_{s} ( t ) {x}_{r} ( t ) = {A}_{s} {A}_{r} cos ( {ω}_{s} t ) cos ( [ {ω}_{s} + {ω}_{d} ] t + φ ) \\ = ( {A}_{s} {A}_{r} / 2 ) \{ cos ( {ω}_{d} t + φ ) + cos ( [ 2 {ω}_{s} + {ω}_{d} ] t + φ ) \}\end{matrix} \qquad ( 4 )$$

In a case where the high-frequency component is removed by the low pass filter 101, the modulation component of a second member in the equation (4) is removed. Then, I(t) which is the I signal after extracting the Doppler frequency component by the low pass filter 101 is expressed by the equation (5).

I(t)=(AₛAᵣ/2)cos(ω_{d}t+φ) (5)

In addition, Q(t) which is the Q signal obtained by delaying the phase by π/2 from the I signal is expressed by the equation (6).

Q(t)=(AₛAᵣ/2)cos(ω_{d}t+φ−π/2) (6)

The I signal represented by the equation (5) and the Q signal represented by the equation (6) are input into the signal obtaining unit 30.

In addition, since the I signal differential value ΔI is ΔI≈dI/dt, and the Q signal differential value ΔQ is ΔQ≈dQ/dt, when each of the equation (5) and the equation (6) is differentiated by the time t, the I signal differential value ΔI and the Q signal differential value ΔQ can be calculated.

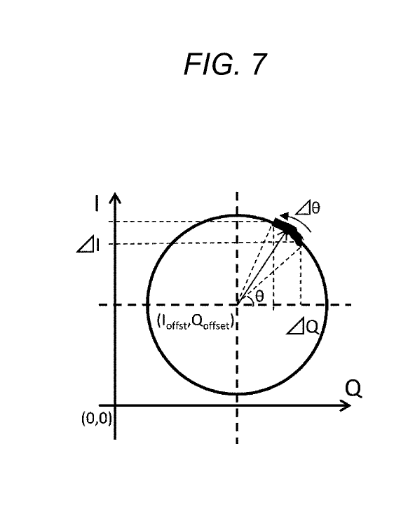

In addition, the angular velocity ω on the I-Q coordinate plane as illustrated in FIG. 7 is ω=dθ/dt.

In addition, since θ=arctan(I−I_{offset})/(Q−Q_{offset}) when I_{offset} can be expressed by a constant defined by an installation condition of the electric wave type biosensor, and Q_{offset} can be expressed by a constant defined by an installation condition of the electric wave type biosensor, the angular velocity ω can be expressed by Expression 1 as follows:

$$ω \approx \frac{( t - {t}_{offset} ) Δ Q - ( Q - {Q}_{offset} ) Δ t}{{( t - {t}_{offset} )}^{2} + {( Q - {Q}_{offset} )}^{2}} \qquad ( Expression 1 )$$

In addition, the IQ norm NRM can be expressed by the equation (7).

NRM=√((I−I_{offset})²+(Q−Q_{offset})²) (7)

In addition, in a case where the band pass filter 102 is not provided, the angular velocity is acquired, for example, by performing time subtraction of the I signal with respect to ΔI and time subtraction of the Q signal with respect to ΔQ.

In addition, the electric wave type biosensor 100 further includes a bio-information extract unit 50 which extracts the bio-information of the living body based on the angular velocity ω calculated by the I-Q norm angular velocity calculation unit 40. The bio-information extract unit 50 extracts the bio-information based on the characteristics of the bio-information to be extracted. For example, in the bio-information extract unit 50, in a case where the frequency component passed through the band pass filter 102 in a previous stage includes the frequency component of both of the heart rate component and respiration, the angular velocity w output by the I-Q norm angular velocity calculation unit 40 is obtained by synthesizing two of the periodical component of respiration and the periodical component of heart rate.

In this manner, in a case where the angular velocity w obtained by synthesizing two of the periodical component of respiration and the periodical component of heart rate is input to the bio-information extract unit 50, the bio-information extract unit 50 can extract the heart rate or the respiratory frequency or the strengths from the heights of each of the peaks by comparing the period of general respiration or heart rate. In this manner, by irradiating the human body surface with the electromagnetic wave, by obtaining a plural pieces of bio-information based on the angular velocity on the coordinate plane of the I signal and the Q signal of the reflected wave, and by extracting a specific bio-information based on the frequency component, such as general heart rate or respiratory frequency, it is possible to obtain various pieces of bio-information at the same time.

In addition, the electric wave type biosensor 100 further includes an output determination unit 80 which determines whether or not the bio-information extracted by the bio-information extract unit 50 is to be output, based on whether or not the size of the angular velocity ω is within a predetermined threshold value; and an external output unit 70 for outputting the bio-information to an external mechanism that uses the bio-information extracted by the bio-information extract unit 50, based on the determination of the output determination unit 80. The output determination unit 80 determines that the bio-information extracted by the bio-information extract unit 50 is to be output in a case where the size of the angular velocity ω calculated by the I-Q norm angular velocity calculation unit 40 or the size of the angular velocity ω estimated by an estimating unit 90 which will be described later, is within the predetermined threshold value. On the contrary, the output determination unit 80 determines that the bio-information extracted by the bio-information extract unit 50 is not to be output in a case where the size or the like of the angular velocity ω calculated by the I-Q norm angular velocity calculation unit 40 exceeds the predetermined threshold value.

Here, the predetermined threshold value will be described with reference to FIG. 7. The size of a circle in FIG. 7 indicates the size of receiving strength in the receiving antenna 21 of the reflected wave, and fluctuates according to a state (distance, inclination of the reflected surface, reflectivity, or the like) of the surface of the living body which is the measurement target TG. In a case where the distance between the Doppler sensor DS and the surface of the living body is d, and a displacement amount Δd of the distance d is expressed by the equation (8).

Δd=λ·Δθ4π (8)

λ is a wavelength (for example, 12.5 mm in a case where the frequency is 24 GHz) of the transmission wave.

In a case where a large fluctuation is generated on the body surface of the living body as the human body moves, the displacement amount Δd of the distance d between the Doppler sensor DS and the surface of the living body increases, and as a result, Δθ also largely fluctuates. Since the Δθ is an angle, it is not possible to determine how much fluctuation is practically performed, for example, when the angle is equal to or greater than 360 degrees. Therefore, the predetermined threshold value (first threshold value) depends on dt which is a sampling interval, but the predetermined threshold value is referred to as the angular velocity ω to the extent that the Δθ does not exceed 360 degrees (+180 degrees and −180 degrees).

In a case where the output determination unit 80 determines that the bio-information is to be output, the external output unit 70 outputs the bio-information to the external mechanism that uses the bio-information, and in a case where the output determination unit 80 determines that the bio-information is not to be output, the external output unit 70 does not output the bio-information to the external mechanism that uses the bio-information. In this manner, in a case of obtaining the bio-information which is accompanied with fine movement included in the reflected wave by irradiating the human body surface with the electromagnetic wave using the Doppler sensor DS, the electric wave type biosensor 100 can stop the output of the bio-information in a case where a large fluctuation that exceeds the predetermined threshold value is generated on the body surface of the living body by determining the case as the size of the angular velocity ω of the I signal/Q signal exceeds the predetermined threshold value. Accordingly, the electric wave type biosensor 100 can output accurate bio-information.

In addition, the electric wave type biosensor 100 can selectively include the estimating unit 90 which estimates the size of the angular velocity based on the data of the angular velocity of a time series, which is calculated by the I-Q norm angular velocity calculation unit 40. In a case where the electric wave type biosensor 100 includes the estimating unit 90, the output determination unit 80 determines whether or not the bio-information extracted by the bio-information extract unit 50 is to be output, based on whether or not the size of the angular velocity ω estimated by the estimating unit 90 is within the predetermined threshold value (first threshold value).

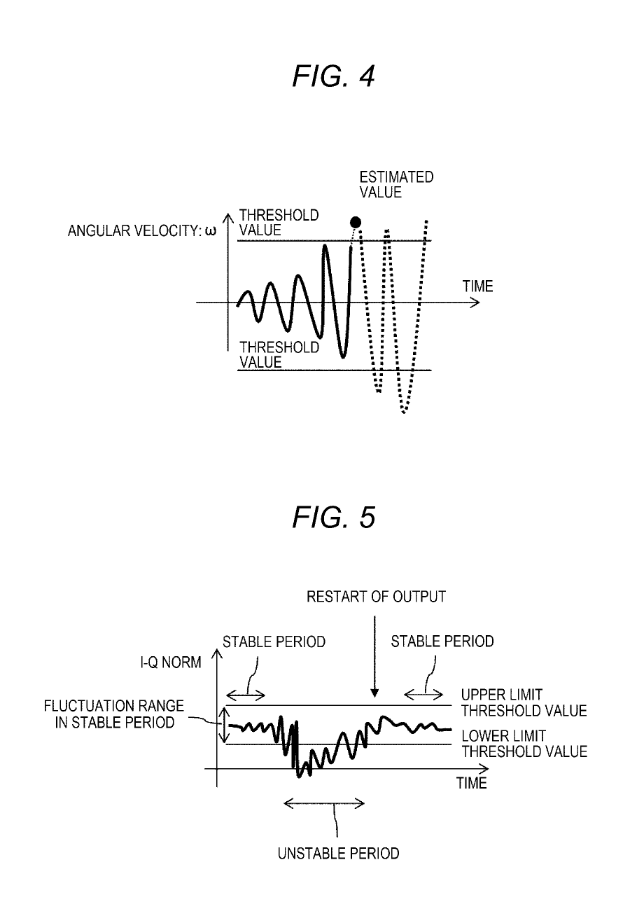

The estimating unit 90 estimates the angular velocity, for example, as illustrated in FIG. 4. In other words, in a case where the change rate of the angular velocity ω gradually increases and the estimated value on a dotted line of the drawing is estimated when the change rate exceeds the predetermined threshold value, by making a decision from the data (part illustrated by a solid line in the drawing) of the angular velocity of the time series calculated by the I-Q norm angular velocity calculation unit 40, it is determined whether or not the bio-information is to be output based on the estimated value. In addition, a method of estimation illustrated in FIG. 4 is an example, and the estimated value may be, for example, a squared value of the angular velocity. According to this, by estimating whether or not the range where the displacement of the surface of the living body can be measured deviates, it is possible to determine whether or not the angular velocity w is practically rapidly output before exceeding the predetermined threshold value, and to output only accurate bio-information without outputting wrong bio-information.

In addition, after the output determination unit 80 determines that the bio-information is not to be output, the output determination unit 80 determines that the bio-information extracted by the bio-information extract unit 50 is output in a case where the IQ norm NRM calculated by the I-Q norm angular velocity calculation unit 40 is within the predetermined threshold value (second threshold value) and in a case where the size of the angular velocity calculated by the I-Q norm angular velocity calculation unit 40 is within the predetermined threshold value (first threshold value). The predetermined threshold value (second threshold value) related to the IQ norm NRM is determined, for example, by the method illustrated in FIG. 5. As illustrated in the drawing, the output determination unit 80 considers a period during which an absolute value of the angular velocity ω is sufficiently small as a stable period, and learns a fluctuation range of the IQ norm NRM in the stable period. For example, the predetermined threshold value (second threshold value) related to the IQ norm NRM can be acquired from a mean value and distribution of the stable period, and as an example, the second threshold value can be a mean value±3σ (σ: deviation). In this case, an upper limit threshold value of the drawing can be a mean value +3σ, and a lower limit threshold value can be a mean value −3σ.

The surface of the living body largely fluctuates, and the angular velocity ω itself also largely exceeds the first threshold value in an unstable period during which the angular velocity ω illustrated in the drawing exceeds the second threshold value (the upper limit threshold value or the lower limit threshold value), and thus, the output determination unit 80 is in a state where it is already determined that the bio-information is not to be output. In addition, when the fluctuation of the IQ norm NRM is converged and the IQ norm NRM is within the second threshold value (the upper limit threshold value or the lower limit threshold value), the unstable period is finished, and the output determination unit 80 determines that the output of the bio-information is to be restarted. In this manner, in a case where deviation from the stable period is determined by the angular velocity ω, and return to the stable period is determined by the IQ norm NRM, a case of determination by the angular velocity w is excellent for determining the stability, but since the angular velocity is not acquired when deviating the range once, the angular velocity is distinguished by using the IQ norm NRM. Accordingly, by restarting the output in a case where the IQ norm NRM is within the predetermined threshold value, it is possible to output accurate bio-information.

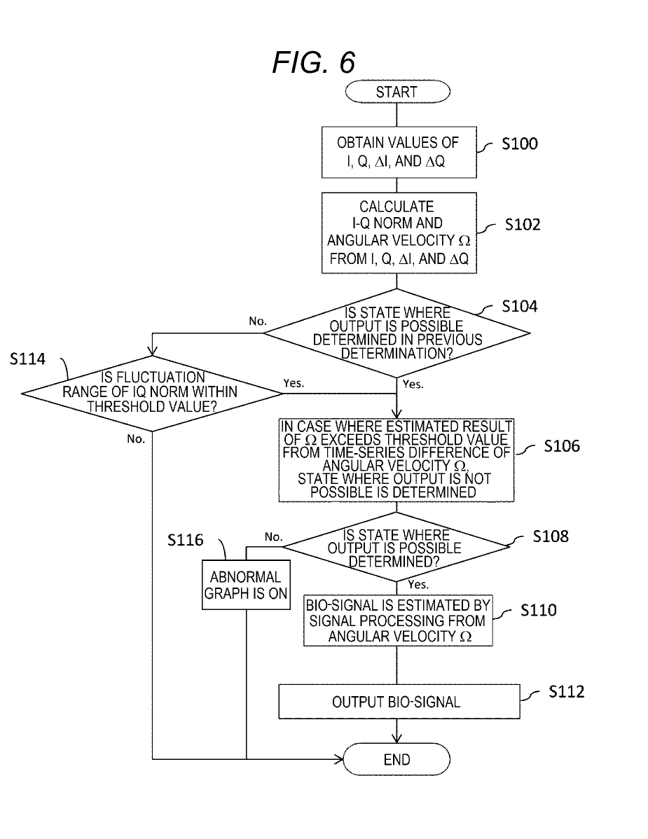

FIG. 6 is a flowchart illustrating a control in the electric wave type biosensor 100. S in the flowchart indicates steps. In S100, the signal obtaining unit 30 of the electric wave type biosensor 100 obtains the I signal and the Q signal which pass through the low pass filter 101 and the I signal differential value ΔI and the Q signal differential value ΔQ which pass through the band pass filter 102. In S102, the I-Q norm angular velocity calculation unit 40 calculates the angular velocity w based on the above-described (Expression 1), and the IQ norm NRM based on the equation (7), from the I signal, the Q signal, the I signal differential value ΔI, and the Q signal differential value ΔQ, which are obtained by the signal obtaining unit 30, and the offset values defined by the installation condition of the electric wave type biosensor 100.

In S104, the output determination unit 80 inspects an abnormal flag which will be described later, and inspects whether or not the bio-information was output in the previous determination (a state where the output is possible, or not). In a case where the output was performed in the previous determination, in S106, the output determination unit 80 is in a state (a state where the output is not possible) where the bio-information is not output in a case where the estimating unit 90 estimates that the angular velocity ω exceeds the predetermined threshold value (first threshold value) by making a decision from the time-series data of the angular velocity ω, that is, in a case where it is assumed that a large fluctuation in which the angular velocity ω exceeds the predetermined threshold value is generated on the surface of the living body.

In S108, the output determination unit 80 inspects whether or not the bio-information is to be output (a state where the output is possible, or not). In a case where the output determination unit 80 determines that the bio-information can be output, that is, in a case where the estimation result of the estimating unit 90 does not exceed the first threshold value in S106, the estimating unit 90 estimates the angular velocity ω again in S110. In addition, in S112, the external output unit 70 outputs the bio-information extracted by the bio-information extract unit 50 to the external mechanism. In a case where the output determination unit 80 determines that the output of the bio-information is not possible in S108, the output determination unit 80 turns on the abnormal flag, and finishes the process after this, and the output of the bio-information is not performed, in S116. The abnormal flag is inspected in S104.

In S104, in a case where the abnormal flag is ON, that is, in a case where the output is not performed in the previous determination, in S114, the output determination unit 80 inspects whether or not the fluctuation range of the IQ norm NRM is within the predetermined threshold value (second threshold value). In a case where the fluctuation range is within the predetermined threshold value, returning to S106, the angular velocity ω is estimated in the estimating unit 90. In addition, in a case where the IQ norm NRM exceeds the predetermined threshold value, the process is finished, and the bio-information is not output.

In this manner, the electric wave type biosensor 100 determines that the surface of the living body largely fluctuates as the angular velocity ω exceeds the predetermined threshold value, and in this case, as the bio-information, such as heart rate, is not output, it is possible to output only accurate bio-information. In addition, as the estimating unit 90 estimates the angular velocity ω, it is possible to rapidly determine whether or not the output is possible. In addition, in a case where the output of the bio-information is stopped once, by determining the restart of the output by the IQ norm NRM, it is possible to output only accurate bio-information.

In addition, the invention is not limited to the exemplified embodiment, and can be realized according to a configuration within a range that does not depart from the contents described in each of the claims. In other words, the invention is illustrated in the drawings mainly particularly regarding the specific embodiment, and is described, but without departing from the technical idea and the range of object, those skilled in the art can add various deformations in the number of components and other specific configurations, with respect to the above-described embodiment.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. An electric wave type biosensor comprising:
an electromagnetic wave irradiation unit which irradiates a body surface of a living body with an electromagnetic wave;
a reflected wave receiving unit which receives a reflected wave obtained as the electromagnetic wave irradiated by the electromagnetic wave irradiation unit and then reflected on the body surface, and obtains an I signal obtained by multiplying the irradiated electromagnetic wave signal and the received reflected signal, and a Q signal obtained by delaying the I signal by a predetermined phase;
an I-Q norm angular velocity calculation unit which calculates an angular velocity and an IQ norm of the I signal and the Q signal, based on the I signal and the Q signal which are obtained by the reflected wave receiving unit;
a bio-information extract unit which extracts bio-information of the living body, based on the angular velocity calculated by the I-Q norm angular velocity calculation unit; and
an output determination unit which determines whether or not the bio-information extracted by the bio-information extract unit is to be output based on whether or not a size of the angular velocity calculated by the I-Q norm angular velocity calculation unit is within a first threshold value.

2. The electric wave type biosensor according to claim 1,
wherein after determining that the bio-information is not to be output, the output determination unit determines that the bio-information extracted by the bio-information extract unit is to be output in a case where the IQ norm calculated by the I-Q norm angular velocity calculation unit is within a second threshold value, and the size of the angular velocity calculated by the I-Q norm angular velocity calculation unit is within the first threshold value.

3. The electric wave type biosensor according to claim 1, further comprising:
an estimating unit which estimates the size of the angular velocity based on data of the angular velocity of a time series which is calculated by the I-Q norm angular velocity calculation unit,
wherein the output determination unit determines whether or not the bio-information extracted by the bio-information extract unit is to be output, based on the size of the angular velocity estimated by the estimating unit and based on whether or not a size of the estimated angular velocity is within the first threshold value.
